(19) United States
(12) Patent Application Publication
(10) Pub. No.: US 2014/0046153 A1
(43) Pub. Date: Feb. 13, 2014

(54) MONITORING VOLAEMIC CONDITION IN A HUMAN OR ANIMAL SUBJECT

(71) Applicant: Stephen Frederick Woodford, Avoca Beach (AU)

(72) Inventor: Stephen Frederick Woodford, Avoca Beach (AU)

(21) Appl. No.: 13/994,866

(22) PCT Filed: Dec. 15, 2011
(86) PCT No.: PCT/AU2011/001612

§ 371 Date: Oct. 9, 2013

Related U.S. Application Data
(60) Provisional application No. 61/424,017, filed on Dec. 16, 2010.

(51) Int. Cl. A61B 5/02; A61B 5/021; A61B 5/00; A61B 5/145; A61B 5/024; A61B 5/1455

(52) U.S. Cl. CPC A61B 5/02028; A61B 5/024; A61B 5/14551; A61B 5/7271; A61B 5/14546; A61B 5/021; A61B 5/742; A61B 5/746
USPC 600/324; 600/481; 600/508; 600/323; 600/309; 600/485

(57) ABSTRACT

A method for processing data to monitor volaemic condition in a human or animal subject includes identifying one or more reference values for each of one or more haemodynamic variables; receiving haemodynamic data representing the one or more haemodynamic variables measured from the subject over time; for at least one of the one or more haemodynamic variables, comparing the haemodynamic data with the respective one or more reference values; and identifying the existence of abnormal volaemic condition in the subject when the comparison indicates a deviation from the one or more reference values for at least one of the haemodynamic variables. The method may be combined with a visual display of haemodynamic data in real time, ideally in conjunction with a visual indicator of ideal reference values.

### FIELD OF THE INVENTION

The present invention relates to a method, system and computer program product for monitoring volaemic condition in a human or animal subject. It relates particularly but not exclusively to a computer-implemented method, system and computer program product for monitoring volaemic variations and particularly, deviation from a reference value, preferably in real time, for use in early detection of an abnormal volaemic condition.

### BACKGROUND TO THE INVENTION

In a normal state of health the human or animal body system continually maintains physiological balance. Even during times of external influence due to disease, drugs, surgical intervention, trauma, cardio pulmonary by pass and the like, the body system auto regulates in order to maintain physiological balance. To achieve this balance, receptors throughout the body work to monitor and adjust haemodynamic variables such as pressure and flow. Traditionally, monitoring patients during surgery and resuscitation involves obtaining blood pressure measurements together with measurements of oxygen saturation, HR, ECG and in the most severe cases, cardiac output. Each of these parameters together with the clinician's assessment of physical signs such as skin colour, skin moistness and temperature can indicate the extent to which a subject's circulation is performing normally.

Hypovolaemic shock is a clinical syndrome that results from a decreased intravascular volume. This may be due to e.g. haemorrhage, plasma or water and electrolyte losses. Typically, hypovolaemic shock leads to inadequate tissue perfusion and this can result in cellular dysfunction and ultimately organ damage.

Hypovolaemic shock is characterised by a loss in intravascular volume that results in decreased preload to the heart. Preload is one of the determinants of stroke volume. When preload is reduced, stroke volume and hence cardiac output falls. In the healthy subject, compensatory mechanisms under neuroendocrine control help to maintain adequate central perfusion despite the fall in cardiac output. However, when compensatory mechanisms fail, systemic vasoconstriction may lead to tissue ischemia, hypoxia and eventually altered cellular function and global organ dysfunction.

Clinical signs indicating the existence of hypovolaemia are somewhat subjective. For blood losses greater than 1500 ml (shock class I), the patient's breathing becomes shallow and rapid (tachypnoea). The appearance of the subject rarely changes for blood losses less than 750 ml. For blood losses from the circulation of between 750 and 1500 ml (shock class II) the skin becomes pale, moist and cool and capillary refill slows. Thus, when pressure is applied e.g. to the nail bed, it will turn white as usual but regaining colour slows to 4 to 5 seconds or longer. This is a non specific indicator of tissue hypoperfusion (resulting from hypovolaemia) but is very sensitive. Typically, the patient also becomes tachycardic with weak pulse or absent pulses. For blood losses from 1500 to 2000 ml (shock class III) systolic pressure falls. This is a late stage manifestation of hypovolaemia. For blood losses from the intravascular circulation in excess of 2000 ml (shock class IV) the patient becomes tachycardic with very weak pulse. Capillary refill is undetectable and the skin becomes pale and moist. Systolic/diastolic pressure becomes very low or undetectable, as does blood pressure.

Typically, in the operating environment or in resuscitation the patient is not conscious and so it is not possible to establish the neurological indicators associated with hypovolaemia such as dizziness, restlessness, anxiety, agitation, confusion and in more severe cases, drowsiness and coma. In some cases urinary output can also be used to indicate the presence of hypovolaemia although like other indicators, urinary output variations are typically not identifiable until the hypovolaemic condition has been exacerbated to a point where there is in excess of 750 ml of volume lost from the vascular circulation.

Currently, hypovolaemic conditions are monitored by checking a range of vital signs of the subject and conducting blood gas analysis to assess the existence of anaerobic metabolism (which produces lactic acidocous). Currently, central venous pressure is taken to be the most reliable indicator of hypovolaemia in patients undergoing surgery and/or resuscitation but this measure alone is inadequate for the purpose of early and reliable identification of hypovolaemia.

It would be desirable to provide an improved approach to monitoring the volaemic condition of a subject such that abnormal volaemic condition can be identified earlier and treated. It would also be desirable to be able to quantify the extent of abnormality.

A reference herein to a patent document or other matter which is given as prior art is not to be taken as an admission or a suggestion that that document or matter was known or that the information it contains was part of the common general knowledge as at the priority date of any of the claims.

### SUMMARY OF THE INVENTION

Viewed from one aspect, the present invention provides a method for processing data to monitor volaemic condition in a human or animal subject, including the steps of: (a) identifying one or more reference values for each of one or more haemodynamic variables; (b) receiving haemodynamic data representing the one or more haemodynamic variables measured from the subject over time; (c) for at least one of the one or more haemodynamic variables, comparing the received haemodynamic data with the respective one or more reference values; and (d) identifying the existence of abnormal volaemic condition in the subject when the comparison indicates a deviation from the one or more reference values for at least one of the haemodynamic variables.

Preferably, the variables are ones which are indicative of intravascular volume variation, and the haemodynamic data is received in substantially real time. The haemodynamic variables may include but are not limited to one or more of Heart Rate (HR), Vascular Resistance (VR)and Stroke Volume (SV). In one example, Vascular Resistance is adjusted by a multiplier to correct for heart rate effects, giving rise to instantaneous (actual) vascular resistance (iVR) as a haemodynamic variable. Other haemodynamic variables may include Central Venous Oxygen Saturation (ScvO2) and Haemoglobin (Hb) concentration. Other haemodynamic variables such Systolic Blood Pressure (SBP), Diastolic Blood Pressure (DBP), Pulse Pressure (PP calculated as SBP-DBP) and Mean Arterial Pressure (MAP) may also be useful in monitoring the volaemic condition of the subject. SBP, DBP, PP and MAP are available from a blood pressure cuff, so maximising the value of data obtained noninvasively. Fractional changes in SBP, DBP and PP may elicit different patterns depending on the cause of the change in pressure.

Other physiological variables such as Base Excess BE and pH may be monitored concurrently to complement information, diagnoses and intervention made possible by the present invention. These additional variables may provide guidance as to other and potentially significant pathology within the subject whilst still permitting achievement of target haemodynamic performance as may be determined by monitoring SV, iVR, HR and the like.

The one or more reference values for at least one of the one or more haemodynamic variables may be derived from actual haemodynamic data obtained from the subject, ideally while the subject is normo-volaemic. Alternatively, the one or more reference values may determined from data obtained from a population of individuals, ideally while normo-volaemic.

In a preferred embodiment, the method includes displaying on a display means a time-based chart representing data values obtained from the subject for at least one of the one or more haemodynamic variables and optionally, an indicator for the respective one or more reference values. The method may also include the step of calculating a ratio of received data value to reference value for at least one haemodynamic variable, and displaying the calculated ratio on a display means. This ratio may be referred to as a “fractional change” in the haemodynamic variable. The method may also include the step of plotting the ratio (or fractional change) over time. This enables the clinician to determine, upon visual inspection, a diversion from “unity” which indicates a change in volumetric index, where values greater than unity indicate over-filling and values less than unity indicate under-filling of the circulation, when compared to the reference valued.

Alternatively/additionally the method may include allocating a score for at least one of the haemodynamic variables based on their values in the data, where the allocated score represents a degree of deviation from one or more reference values for that haemodynamic variable. In one or more embodiments, the score is re-calculated as data is received and may be represented visually for the at least one haemodynamic variables as the score changes over time, indicating changes in the haemodynamic function of the subject. The method may also include determining the extent of abnormal volaemic condition according to one or more allocated scores. A score may be allocated for a plurality of haemodynamic variables and the extent of abnormal volaemic condition may be determined according to a combined score from the allocated scores, e.g. a sum-total of allocated scores. Thus, in one or more embodiments, the method may include determining an extent of abnormal volaemic condition according to a divergence of a calculated ratio from unity which represents concordance between the received value for the haemodynamic variable, and the corresponding reference value.

In one or more embodiments, the method includes the step of determining a score indicating volaemic index by (i) calculating a sum total of (a) each ratio value calculated for each haemodynamic variable known to decrease with volume loss, plus (b) the inverse of each ratio value calculated for each haemodynamic variable known to increase with volume loss, and (ii) dividing the calculated sum total by the number of haemodynamic variables considered in step (i).

The method may further include determining a reliability indicator for indicating the reliability of a score, where a score calculated using more variables has a higher reliability than a score calculated using fewer variables. Alternatively/additionally an allocated score may be adjusted to have greater or lesser influence on determining the extent of abnormal volaemic condition, wherein the adjustment may be based on one or more of sensitivity of a haemodynamic variable to volume changes; age, fitness, gender, weight or condition of the subject; and therapies being administered to the subject. In one embodiment, the adjustment is a weighting coefficient which is determined automatically by a processing device using reference data from a population of individuals, and subject-specific data provided to the processing device. In another embodiment, the adjustment is a weighting coefficient which is determined on a case by case basis by a clinician. A hybrid of the weighting coefficients may also be used.

The method may also include the step of selecting automatically a course of action targeted at restoring one or more of the haemodynamic variables toward the one or more reference values by interrogating a collection of pooled data indexing haemodynamic variables and therapeutic effectors obtained from a population of individuals. As an adjunct the method may include providing clinical guidance to physicians. Alternatively/additionally, the method may include the step of providing control signals to infusion pumps or other devices for an automated method of titration of the selected therapy to the subject in a closed loop feedback system aimed at restoring haemodynamic performance to target values. The target values may be selectable from available reference values that are indexed to subject-specific factors such as e.g. age, gender, health condition, etc.

Viewed from another aspect, the present invention provides a computer program product storing instructions for controlling a processing device to perform a method for monitoring an abnormal volaemic condition in a human or animal subject, wherein the instructions cause the processing device to: (a) receive one or more reference values for each of one or more haemodynamic variables; (b) receive haemodynamic data representing the one or more haemodynamic variables measured from the subject over time; (c) for at least one of the one or more haemodynamic variables, compare the haemodynamic data with the respective one or more reference values; and (d) identify the existence of abnormal volaemic condition when the comparison indicates a deviation from a reference value for at least one of the haemodynamic variables.

The instructions may cause a processing means to calculate one or more reference values for at least one of the one or more haemodynamic variables using actual haemodynamic data previously obtained from the subject, ideally while normo-volaemic. Alternatively/additionally, instructions cause the processing means to receive one or more reference values from memory means or a database in communication with the processing device, wherein the one or more reference values has been determined using data obtained from a population of normo-volaemic individuals.

In a preferred embodiment, the instructions cause the processing means to generate a display signal for causing a display means to present a time-based chart of data values representing at least one of the one or more haemodynamic variables and optionally, an indicator for the respective one or more reference values.

The instructions may further cause the processing means to calculate, for at least one haemodynamic variable, a ratio of actual data value to one or more reference values; and display the calculated ratio on a display means. Alternatively/additionally, the instructions may cause the processing means to calculate a score for at least one of the haemodynamic variables in the data, the calculated score representing a deviation of a data value from a reference value; and estimate the extent of abnormal volaemic condition according to one or more calculated scores. The instructions may further cause the processing means to calculate a score for a plurality of haemodynamic variables and to estimate the extent of abnormal volaemic condition by combining the calculated scores, e.g. by calculating their sum-total. The sum-total may be a weighted sum-total according to the relative importance of one or more of the variables.

In one or more embodiments, the instructions cause the processing means to apply a weighting coefficient to one or more of the calculated scores, wherein the weighting coefficient is selected for a particular haemodynamic variable based on one or more of sensitivity of a haemodynamic variable to volume changes; age, fitness, gender, body mass or physiological condition of the subject; and a therapy being administered to the subject. The instructions may cause the processing means to select automatically, the weighting coefficient applicable to a haemodynamic variable. Alternatively, the instructions may enable the processing means to receive an input from a clinician which indicates the weighting coefficient applied to a score calculated for a haemodynamic variable.

In one or more embodiment, the instructions enable the processing means to interrogate a database indexing haemodynamic variables and therapeutic effectors and to identify automatically a course of action targeted at restoring one or more of the haemodynamic variables toward a reference value, and generating a user interface signal configured to cause a user interface to present the identified course of action to a user. Viewed from yet another aspect, the present invention provides a method of representing visually a state of the circulation of a human or animal subject, including the steps of: (a) charting on a display means in substantially real time, data obtained from the subject representing one or more haemodynamic variables of the subject; and (b) charting on the display means a reference value indicative of substantially normal values for one or more of the charted haemodynamic variables.

In an embodiment, charting the data obtained from the subject includes presenting time-elapsed changes in the data values. The method may also include the step of calculating deviation of the data obtained from the subject from a relevant reference value, and/or the step of charting the calculated deviation on the display means. In one example, the deviation may be calculated as an average or a rolling average of values occurring in a finite time period.

Viewed from another aspect still, the present invention provides a system for monitoring an abnormal volaemic condition in a human or animal subject, the system including: (a) at least one processing means for executing steps of the above method, or executing the instructions described above; (b) a user interface including display means for displaying haemodynamic data and/or a ratio of actual to reference values for one or more haemodynamic variables; and (c) a data interface for receiving data representing the one or more haemodynamic variables measured from the subject.

The system may also include an alert driver for generating an alarm or communicating an alert (e.g. to a paging device or other mobile/hand held unit) when a score exceeds a threshold value. In one or more embodiments there may be software in the form of an “app” or the like installed on or accessible by a smartphone or other handheld device. Once paired with devices at the subject's point of care, the smartphone/handheld device is able to display mapped/charted data to a clinician while away from the subject, as well provide an alert generated by the processing means when the data trends outside of safe territory.

### BRIEF DESCRIPTION OF THE DRAWINGS

Embodiments of the invention will now be described by reference to the accompanying drawings. It is to be understood that the drawings are provided for the purpose of describing features and alternative arrangements for the invention only, and do not limit the scope of the invention as defined in the claims appended hereto.

FIG. 1 is a flow chart outlining steps in a method for monitoring abnormal volaemic condition according to an embodiment of the present invention.

FIG. 2 is a chart of data values for HR, SV, SW and Scv0₂ as they vary with time.

FIG. 3 is a flow chart showing steps in a method for monitoring volaemic condition according to a further embodiment of the present invention.

FIG. 4 is a chart representing deviation of HR, SV and iVR from reference values, according to another embodiment of the present invention.

FIG. 5 is a chart showing variation in time of a “Hypovolaemia Score” calculated as a combined score allocated to data for each haemodynamic variable, representing the extent of deviation from a reference point.

Table 1 is an example of a scoring regime according to an embodiment of the present invention.

Table 2 is an example of another scoring regime according to an embodiment of the present invention.

FIG. 6 is a schematic illustration showing components of a system for monitoring volaemic condition, according to an embodiment of the present invention.

FIG. 7 is data pertaining to Example B.

FIG. 8 is further data pertaining to Example B.

FIG. 9 is a schematic illustration showing cyclical variation in the intravascular volume of a patient.

### DETAILED DESCRIPTION

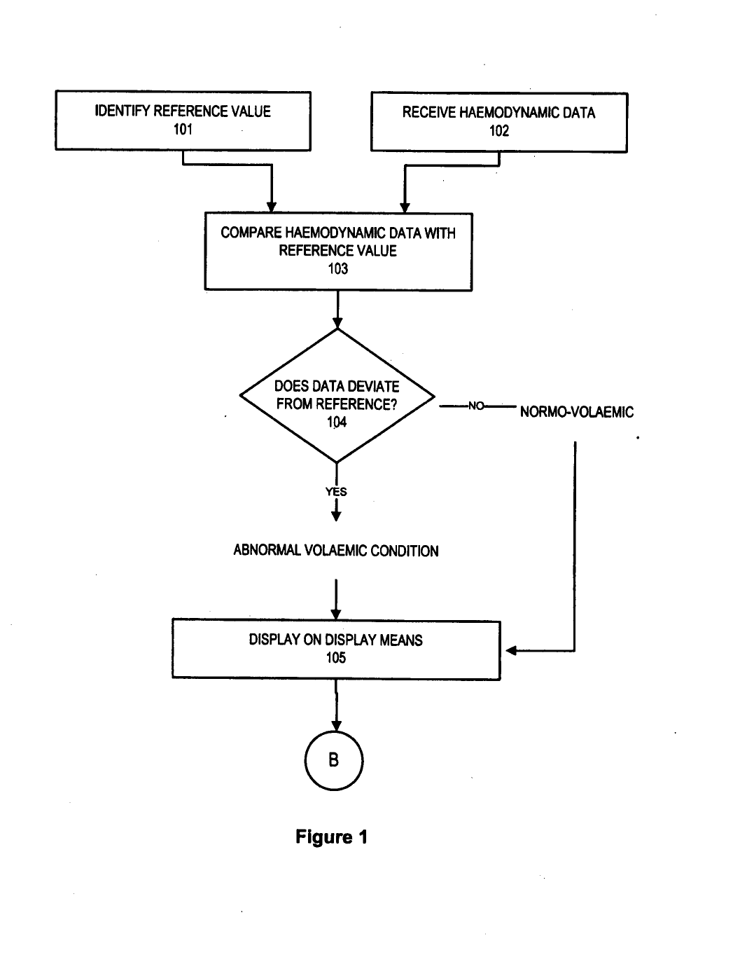

Referring firstly to FIG. 1, there is shown a flow chart outlining steps in a method for monitoring abnormal volaemic condition in a human or animal subject. In a step 101 a reference value is identified for each of one or more haemodynamic variables being monitored. It is to be understood that the term “reference value” may refer to a single value (e.g. HR of 70 bpm) or to a range of acceptable reference values (e.g. HR of 65 to 75 bpm). A single reference value may be calculated as an average of “normal” values measured from the subject or another individual, or a group of individuals.

In a step 102 haemodynamic data representing one or more haemodynamic variables measured from the subject over time are also received. Preferably, the haemodynamic data are received in real time or in substantially real time so that the volaemic condition of the subject can be monitored and therapies can be administered immediately when there is detection of an abnormal condition.

Although a single haemodynamic variable may provide some indication as to the volaemic condition of the subject, it is more desirable to use a collection of different haemodynamic indicators to ascertain the volaemic condition of the subject. In a preferred embodiment, the haemodynamic variables which are monitored and for which data is received include HR, SV and, in a preferred embodiment, iVR or Elastance. In other embodiments, other variables such as SvO₂ and Hb may be monitored.

In the clinical setting, existing monitoring devices typically provide an indicator known as “Systemic Vascular Resistance” (SVR). The inventor has found use of this parameter to be somewhat misleading. The inventor of the instant application has observed, by employing various methods of representing visually a state of the circulation of various human subjects, that SVR is not an accurate indicator of the resistance of the circulation to blood flow.

By definition, flow is a term that represents volume per unit of time. The “resistance” of the circulation to deformation (arising from blood flow) cannot be a time-dependent variable so, calculating the “resistance” to deformation using flow does not make sense. Instead, the inventor uses two alternative determinants of pressure: (i) distending volume; and (ii) the property of the circulation that resists deformation. This gives rise to a solution for flow in the circulation (based .on ohms law) which says:

SVR=iVR/HR (Equation 1)

where SVR is Systemic Vascular Resistance, iVR is Instantaneous Vascular Resistance and HR is Heart Rate. However, since SVR is measured in SI units using a multiplier of 79.9, calculating iVR as the product of SVR and HR requires correction by dividing by 79.9. The beneficial effect of regarding iVR instead of SVR is evident in Example A.

In a patient developing tachyarrythmia before induction of anaesthesia, a graph of the variables SV, SVR, HR and iVR revealed that iVR mirrored the drop in SV as a mechanism to modulate pressure and prevent fluctuations of pressure in the circulation.

In a step 103, the received haemodynamic data is compared with a reference value for at least one of the haemodynamic variables. Since it is desirable to use a collection of different haemodynamic indicators to ascertain the volaemic condition of the subject, it is preferred that two, more preferably three or four or more different variables are compared with relevant reference values and in a preferred embodiment, allocated a score (see below) based on deviation from reference. Additional variables such as Central Venous Oxygen Saturation (ScvO2), BE and pH may be used, particularly when technology exists for these variables to be measured in substantially continuously and in real time and at the subject's point of care.

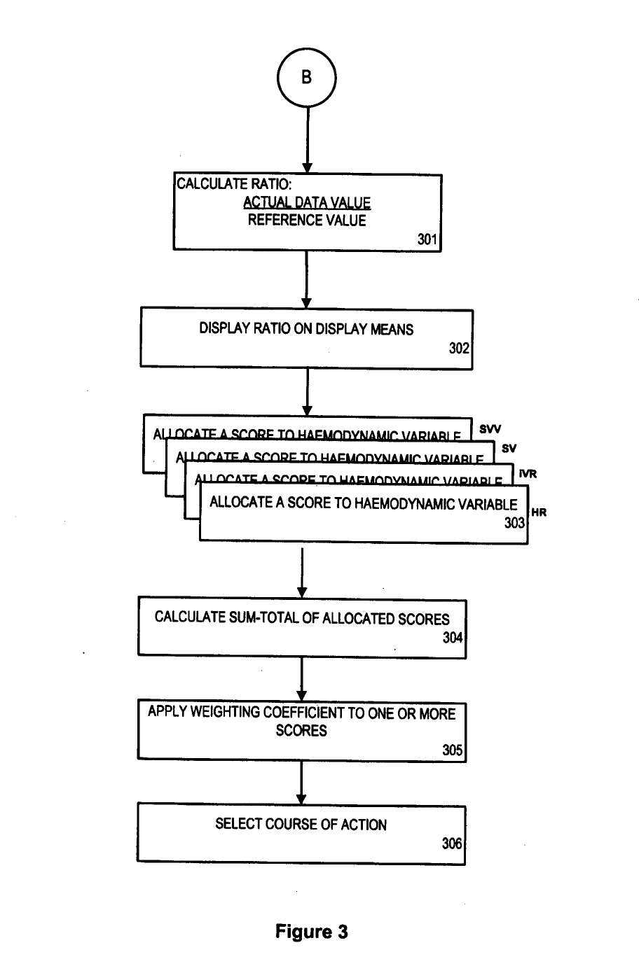

In a step 104, the method involves identifying the existence of an abnormal volaemic condition in the subject when the comparison indicates that the data values deviate from the reference value for at least one of the haemodynamic variables, preferably where there is deviation for at least two of the variables and more preferably where there is deviation for at least three of the haemodynamic variables. The specificity of the method improves when more haemodynamic variables (which are known to be indicators of intravascular volume variation or volaemic index) are observed to deviate from a reference value that indicates substantially normal volume.

Where there is substantially no deviation from the reference value, the subject can be considered “normo-volaemic”. Where there is deviation between the data values and the reference value, then the subject's volaemic condition can be considered “abnormal”. Where an abnormal volaemic condition is identified, the method may further include the optional steps commencing at “B” (FIG. 3).

In a preferred embodiment, the reference value for at least one, and more preferably for all of the haemodynamic variables utilised by the inventive method, is derived from haemodynamic data obtained from the subject while the subject has substantially normo-volaemic condition. For instance, where the subject is undergoing elective surgery and requires general anaesthesia, it is desirable to monitor the haemodynamic variables for that subject prior to induction of anaesthesia, while the subject is in a substantially normo-volaemic state (i.e. neither volume depleted nor overloaded). The actual data collected from the subject can then be used to provide a reference value or a range of reference values with which data collected during anaesthesia may be compared, according to the inventive method. The reference values may be a mean, median or average of values taken over a period of time.

However it is not always possible to monitor the subject while normo-volaemic. Thus, in certain scenarios it is necessary to utilise reference values obtained from other sources. Accordingly, in an alternative embodiment reference value(s) for one or more haemodynamic variables is determined from data that has been obtained previously from a population of normo-volaemic individuals. Ideally, the reference value(s) are determined from data obtained from a population of individuals having relevant or similar physiological characterisation as the subject. Thus, the reference value(s) may be obtained from data relating to sub-sets of a general population, e.g. populations having the same gender, age, body mass, medical history, or a hybrid of these. This enables clinicians to tailor treatment toward the physiological needs of individual patients or patient cohorts. This is an improvement upon the current approach which applies a single physiological paradigm to all patients irrespective of their age, gender, body mass and other variables which cause specific changes in how pressure and flow are regulated.

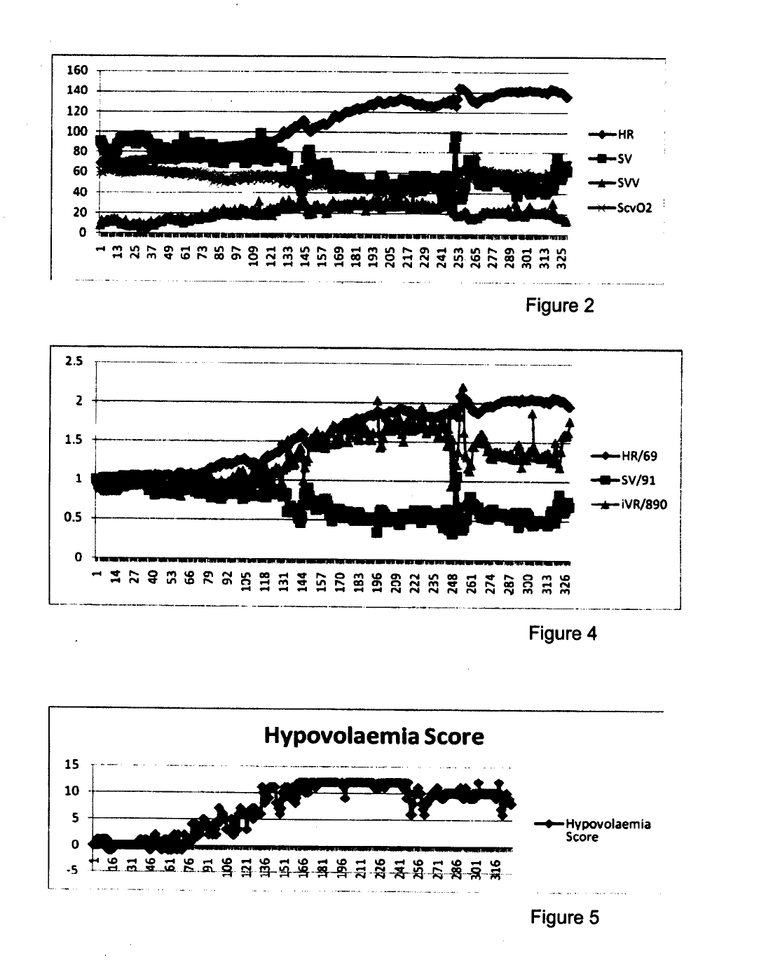

In a preferred embodiment the method includes the step 105 of displaying on a display means a time-based chart representing data values for at least one of the one or more haemodynamic variables. One such plot is shown in FIG. 2.

FIG. 2 is a time-based plot representing the changes in four haemodynamic variables measured from a subject over time, in twelve second intervals. The variables monitored are HR, SV, stroke volume variation (SW) and (Scv0₂). The chart of actual data values is somewhat useful in that it can be used to identify when changes in these parameters occur. However, the inventor has discovered that it is of greater utility to chart the extent to which these variables deviate from reference values, in order to make an assessment as to the changing and potentially abnormal volaemic state of the subject.

Thus, in one embodiment the method further includes the steps illustrated in FIG. 3. In a step 301, a ratio is calculated for at least one of the haemodynamic variables and preferably for all of the haemodynamic variables for which data is available where the actual data value is the numerator and the reference value is the denominator. Calculation of the ratio may be performed instantaneously, i.e. for a moment in time and recalculated periodically, e.g. every 5, 10, 12, 15, 20, 30, 60 or 180 seconds, or 5 minutes although other time periods are contemplated. Alternatively, the ratio may be calculated based on an average of data values obtained over a time period, e.g. 5, 10, 12, 15, 20, 30, 60 or 180 seconds or 5 minutes. Where an average of values is used, the average may be a rolling average measured over a moving window of time of 5, 10, 12, 15, 20, 30, 60 or 180 seconds or 5 minutes.

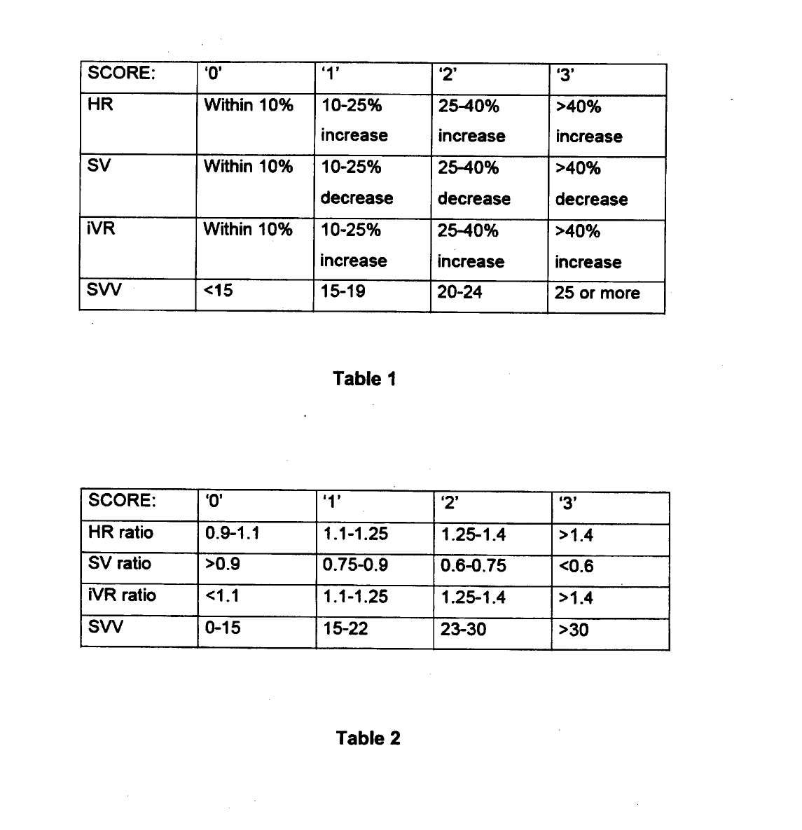

In a preferred embodiment the calculated ratio for at least one and preferably for a plurality of the haemodynamic variables is presented on a display means in a step 302. An example of a time-based chart representing calculated ratios is shown in FIG. 4 (HR, SV and iVR). At t=1 each of these haemodynamic variables is substantially equivalent to the reference value (ratio=1) indicating that the subject is in a substantially normo-volaemic state. At approximately t=79, the variables begin to change; HR and iVR increase and SV decreases.

A clinician can easily determine the extent to which these variables have deviated from the reference value by regarding the extent to which each calculated ratio has diverged from unity. The ratio value 1.5 indicates a 50% deviation, ratio value 2 represents a 100% deviation, etc. Similarly, the ratio value 0.5 represents a minus 50% deviation from the reference value (e.g. a stroke volume decrease of 50% is shown at ratio value 0.5 in FIG. 4). Thus, by charting the ratio for each of the variables on a single display, the percentage deviation from a given starting point (i.e. the reference value) can be quantified easily and simultaneously for all variables, upon visual inspection.

In one embodiment, a score can be assigned to each of the haemodynamic variables, based on the extent to which the data values for that variable deviate from the reference value. This is shown in step 303 in FIG. 3. The score may be determined based on the haemodynamic data directly, although in one embodiment, the score is calculated using the ratios calculated in step 301.

Table 1 shows an example of a scoring system for each of the variables: HR, SV, iVR and SW. Thus, where HR is within 10% above the reference value, the allocated score is 0; where HR is 10-25% above the reference value the allocated score is 1; where HR is 25-40% above the reference value, the allocated score is 2 and where HR is greater than 40% above the reference value, the allocated score is 3. For SV, where the actual value is within 10% of the reference value, the allocated score is 0; where the actual value is 10-25% below the reference value, the allocated score is 1; where the actual value is 25-40% below the reference value, the allocated score is 2 and where the actual value is greater than 40% below the reference value, the allocated score is 3. For iVR, where the actual value is within 10% above the reference value, the allocated score is 0; where the actual value is 10-25% above the reference value, the allocated score is 1; where the actual value is 25-40% above the reference value, the allocated score is 2 and where the actual value is more than 40% above the reference value, the allocated score is 3.

Scores in Table 1 are based on observations that when a subject is hypovolaemic, there is increase in HR, increase in iVR and decrease in SV. For subjects being mechanically ventilated, SW also increases. SW scores can be absolute since SW is already a referenced value (calculated as the percentage variation in SV between inspiration and expiration). Accordingly, absolute SW values may be used to allocate scores for that haemodynamic variable. Thus, for SW a value of less than 15 may be allocated a score of 0; a SW value between 15 and 19 may be allocated a score of 1; a SW value between 20 and 24 may be allocated a score of 2; and a SW value of 25 or more may be allocated a score of 3.

Each of these scores may be considered in isolation in determining the volaemic state of the subject. However, in a preferred embodiment the individual scores for each variable are combined to provide an overall indicator of the subject's volaemic condition. In one embodiment, there may be a treatment protocol known to apply when a combined hypovolaemic score is within particular ranges. For instance, if the combined score is 3 to 5 (from a possible total of 12) protocol may be to give fluid and monitor; if the combined score is 6 to 8 the protocol may be to give fluid, repeat chest x-ray urgently and have the resident doctor attend; if the combined score is 9 or greater the protocol may be to notify the intensivist who must decide if the subject is to be returned to theatre for operation/re-operation.

In an alternative method, a volaemic index is determined by calculating a sum total of (a) each ratio value calculated for each monitored haemodynamic variable known to decrease with volume loss; plus (b) the inverse of each ratio value calculated for each monitored haemodynamic variable known to increase with volume loss; and then dividing the calculated sum total by the number of haemodynamic variables considered in calculating the sum total. This removes the need for expert input in determining the relative value of each score allocated in the previously described scoring scheme. For example, a volaemic index, as a measure of vascular emptiness, may combine the fractional change in Stroke Volume (SV_{fc}) and the fractional change in Elastance (E_{fc}) as follows:

Volaemic index=[SV_{fc}+1/E_{fc}]/2 (Equation 2)

One could also combine the fractional change in Stroke Volume (SV_{fc}) and Elastance (E_{fc}) with the fractional change in Heart Rate (HR_{fc})by averaging as follows:

Volaemic index=[SV_{fc}+1/E_{fc}+1/HR_{fc}]/3 (Equation 3)

Calculating these indices enables Clinicians to track vascular filling; the index falls below unity as the circulation empties, and exceed unity if the circulation is full.

One may alternatively/additionally relate multiple indicators to each other, combining them to emphasise the magnitude of the divergence from a normo-volaemic state associated with a ration value equal to unity. For Example, by continuously monitoring SV_{fc} and E_{fc} one can continuously track SV_{fc}/E_{fc}. The derivative of this fraction may then be calculated in real time and the value may be charted. Changes in the combined fraction SV_{fc}/E_{fc} over time can be used as an early indicator of volume changes and may be useful in administering therapy. It may also be used to obtain an early indication of a subject's response to intervention. An example is provided in Example B.

Because the combined average score takes account of the number of variables considered in determining the score, it can be compared between subjects, even in the case where only 2 haemodynamic variables have been monitored in subject A and 4 haemodynamic variables have been monitored in subject B. However the specificity and hence reliability of the average score for subject and A will be less than that for subject B.

In a preferred embodiment, the method includes determining an indicator of reliability and presenting this to the clinician together with a score so that the score is not considered in a vacuum but in the context in which it has been obtained.

The reliability indicator generally takes account of the number of variables considered in calculating a score but may also take into consideration the origin of the reference value(s) and e.g. the size of the population used to calculate the reference value(s), and/or the extent to which individuals in that population have physiological characteristics common to the subject (e.g. age, gender, body mass). The reliability indicator may be e.g. low, medium or high, or provide a colour indicator (e.g. green for reliable, red for nor reliable, orange for moderately reliable). Alternatively, the reliability indicator provided may be a percentage value of reliability of the score. The reliability indicator may also influence a weighting coefficient.

FIG. 5 shows a chart of “Hypovolaemia Score” varying with time, where the score is calculated using the scoring regime of Table 1. It is to be understood however that many different scoring regimes may be adopted. The example shown in Table 1 is merely one example to illustrate how the volaemic condition of a patient can be quantified. An alternative scoring system is shown in Table 2, where ratio values (rather than percentages) are indexed to a score value. It is to be understood that the scoring ranges may be larger or smaller and increased or decreased. Also, the number of and separation between quantisation levels of deviation may be increased/decreased such that each haemodynamic variable has a score from 0 to 10, 20, 30, 40, 50 etc.

The haemodynamic variables need not all be scored on a range of 0 to 3. One variable which has particularly high sensitivity may cover a wider range (e.g. 1 to 10) to increase the effect of that variable in calculating the sum-total score used to assess the extent of a subject's abnormal volaemic condition. This may be particularly important when dealing with variable values obtained non-invasively as they are likely to have less accuracy than variable values obtained using more invasive techniques.

The accuracy and value of the inventive method and the scoring/ratio analysis approaches discussed herein may be improved by accessing large data sets which index demographic information such as age, gender, body mass index (BMI), health status etc. Indeed indexed data sets containing haemodynamic data obtained from vast quantities of “healthy” subjects as well subjects known to have experienced haemodynamic dysfunction such as haemorrhage, tamponade and sepsis, provide a mechanism for improving diagnostic guidance and intervention. Real time streaming of such data (e.g. via the Internet or other “cloud computing” environment drawing together data from separate databases throughout the world) into diagnostic modules may give rise to constantly improving patient outcomes in the clinical setting.

Preferably, an allocated score may be adjusted to have a greater or lesser influence on determining the extent of abnormal volaemic condition in a subject. The adjustment may be based on one or more of e.g. sensitivity of one of the haemodynamic variables to volume changes (e.g. a particular volume change may have more influence on HR than SV); the age, fitness, gender, body mass or health status of the subject and one or more therapies being administered to the subject. Adjusting the importance of one or more haemodynamic variables gives the method flexibility in dealing with drugs or other therapies (fluid etc.) that are administered, e.g. to stabilise certain physiological parameters. Preferably, the adjustment is achieved by applying a weighting coefficient to one or more of the haemodynamic variables (and therefore the scores calculated for the respective variable).

In one embodiment, the method is performed using a computer processing apparatus or a device including a computer processing apparatus. Thus, the weighting coefficient may be determined automatically by a processing means in the apparatus using e.g. reference data obtained from a population of individuals and the coefficient may be selected based on subject specific data (such as age, gender, body mass, etc.) which is provided to the processing device. Alternatively, the adjustment may involve use of a weighting coefficient which is determined on a case by case basis by a clinician overseeing care of the subject. In an alternative embodiment, the adjustment is a hybrid of weighting coefficients obtained automatically by the processing device and determined by the clinician.

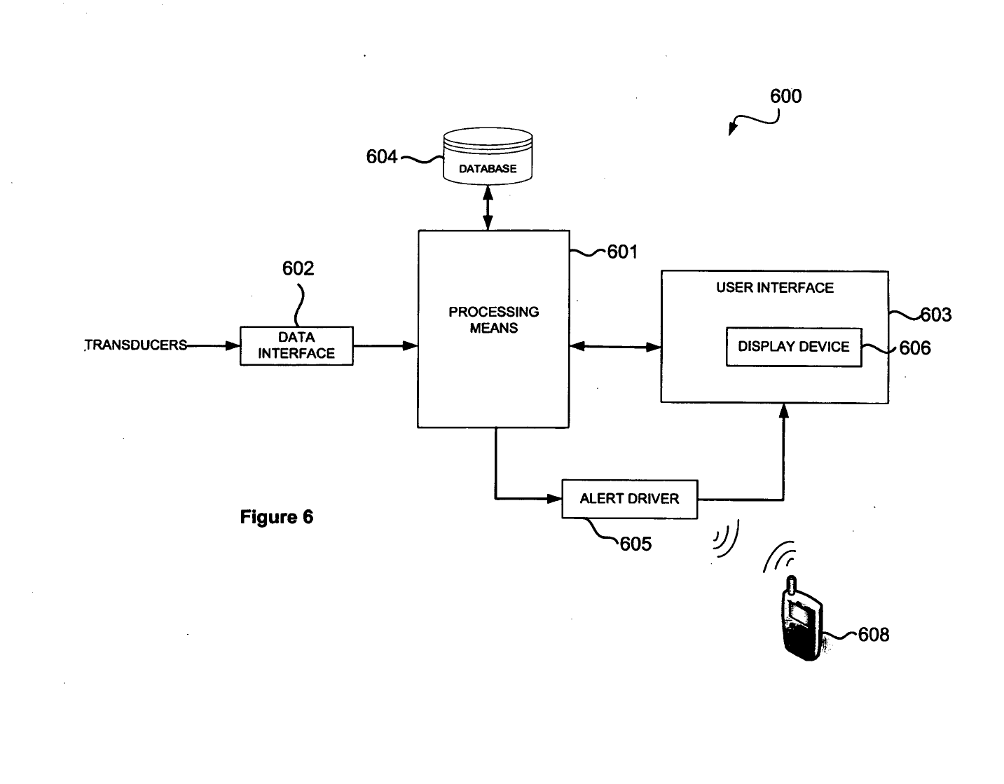

In one embodiment, the method includes the step 306 of selecting a course of action to be administered which is targeted at restoring one or more of the haemodynamic variables toward the reference value. Preferably, this ultimately achieves the outcome of restoring a substantially normal volaemic condition within the subject. Selection of the appropriate course of action may be achieved by interrogating a collection of population data stored in a database and which indexes various haemodynamic variables against therapeutic effectors. Ideally, the selection is performed automatically by a processing apparatus performing other steps of the method, or a device communicating with the processing apparatus performing those steps. An example of a system incorporating such a processing apparatus is illustrated in FIG. 6. Ideally, therapy selected by the processing means 601 is informed by other data pertaining to the subject such as e.g. fluid added to the circulation, drugs administered, surgical interventions and the like, since these factors combine with physiological controls indigenous to the subject to influence haemodynamic function.

Now referring to FIG. 6 components of a system 600 for monitoring an abnormal volaemic condition in a human or animal subject are shown. The system includes processing means 601, a data interface 602 and a user interface 603. Preferably, the system is also in communication with or incorporates database 604 and in one embodiment includes an alert driver 605. The system 600 may run on a stand alone patient monitoring device (e.g. for use in ambulatory settings) or maybe incorporated into other devices used in the clinical setting.

The processing means 601 may incorporate or have access to a computer program product storing instructions for controlling the processing means to perform a method for monitoring an abnormal volaemic condition in a human or animal subject, where the method substantially reflects the steps of the method as outlined in FIGS. 1 and 3 and the description of those Figures. Ideally, instructions stored on the computer program product cause the processing means 601 to receive one or more reference values for each of one or more haemodynamic variables and to receive haemodynamic data measured from the subject over time for at least one of the one or more haemodynamic variables. The instructions also cause the processing means 601 to compare the haemodynamic data with the respective reference value and, identify the existence of an abnormal volaemic condition where the comparison indicates a deviation from the reference value for at least one of the haemodynamic variables. Existence of an abnormal volaemic condition is identified with more certainty when a plurality of the haemodynamic variables deviates from the relevant reference value.

In one embodiment, the computer program product includes instructions causing a processing means (either the processing means 601 of FIG. 6 or an alternative processing means communicating with the system 600) to calculate reference value(s) for at least one of the one or more haemodynamic variables, using actual haemodynamic data previously obtained from the subject while normo-volaemic. There are advantages in using the subject's own data for the reference value(s) for determining the extent to which that subject is suffering from an abnormal volaemic condition in that the behaviour of each individual's circulation is unique to that individual and so utilising reference values which reflect “normality” is more clinically relevant where the reference values have been obtained from the subject himself or herself.

However, as discussed previously, that is not always possible, e.g. in the emergency setting. Thus in another embodiment the computer program product includes instructions causing the processing means 601 (or an alternative processing means) to receive the reference value for at least one of the one or more haemodynamic variables from memory means which is in communication with the processing device. The memory means may be e.g. a database 604 as illustrated in FIG. 6. The database may be in direct communication with the processing means. Alternatively, it may be a networked storage device accessible on a local area network (LAN) or on a wide area network (WAN) such as the Internet or accessed via cloud computing. Ideally, database 604 contains one or more look up tables or the like relating haemodynamic data obtained from a population of normo-volaemic individuals and that data is accessed to identify a reference value or a range of reference values which are appropriate for the subject in a normo-volaemic state. The reference values may be selected from pooled data in the database according to specific characteristics such as gender, age, medical history, body mass or a combination of these, since these factors influence the behaviour of the circulation in the human body.

Preferably, the computer program product includes instructions causing the processing means 601 to generate a display signal for causing a display means 606 to present a time-based chart of data values for at least one of the one or more haemodynamic variables (see for example the chart of FIG. 2). An indicator for the respective reference value may also be provided.

In a further preferred embodiment, the computer program product contains instructions causing the processing means to calculate, for at least one of the haemodynamic variables and preferably for a plurality of haemodynamic variables, a ratio of actual data value to reference value and displaying the calculated ratio on the display means 606 (see for example the chart of FIG. 4). The calculation of the ratio may be e.g. instantaneous or time averaged over a finite time period or a rolling window of time, e.g. 5, 10, 12, 15, 20, 30, 60 or 180 seconds or longer intervals.

In one embodiment, the computer program product includes instructions causing the processing means 601 to calculate a score for at least one of the haemodynamic variables in the data, where the calculated score represents the deviation of the data value(s) from the reference value for that variable. Tables 1 and 2 indicate examples of scoring regimes, although these are examples only and are not to be taken as limiting the range of scores or scoring regimes that may be adopted. Further, it is to be noted that the scoring regimes of Tables 1 and 2 are targeted only at detecting the hypovolaemic state. Other scoring regimes may be adopted to indicate where there is overfilling of the circulation. The instructions preferably cause the processing means to estimate the extent of abnormal volaemic condition according to one or more of the calculated scores and preferably, according to a combined score for a plurality of haemodynamic variables.

In one embodiment, the computer program product includes instructions causing the processing means 601 to apply a weighting coefficient to one or more of the calculated scores, wherein the weighting coefficient is selected for a particular haemodynamic variable based on one or more of sensitivity of the haemodynamic variable to volume changes; age, fitness, gender, body mass or condition of the subject and a therapy being administered to the subject. Preferably, the instructions cause the processing means to select automatically the weighting coefficient applied to a calculated score, although in an alternative arrangement, the instructions may cause the processing means to receive an input, via user interface 603, from a clinician who selects the weighting coefficient applied to a score calculated for a particular haemodynamic variable.

Preferably, processing means 601 executes instructions to detect an evolving pattern e.g. of volume loss in the circulation. In such configuration, the system may be able to detect hypovolaemia at an early stage which may be correctable e.g. with 50 ml of fluid. This enables intervention before the patient deteriorates further and begins decompensating potentially reducing complication rates, intensive care unit recovery times, admissions to intensive care and resulting in earlier discharge from hospital. Also, by accessing a sufficiently detailed database indicating the haemodynamic effects of fluid or drugs, the processing means may be configured to calculate and titrate the therapy automatically while anticipating and adjusting for the impact of the therapy on the subject. For example, if the processing means knows that amiodarone is being administered, it will pre-correct for the expected drop in blood pressure by increasing the rate of noradrenaline administered.

The processing means 601 may receive a range of different inputs via an interface 602. These inputs may be derived from transducers such as the kind used to continuously monitor S_{cv}O₂, Haemoglobin (Hb) and other variables indigenous to the subject. Other inputs may include e.g. infusion pump data which informs the processing means 601 as to each volume of fluid given to the subject, as well as the time and rate of delivery. Drug administration data may be received by the processing means at another input. These provide additional data that may be used to detect e.g. volume deficiencies in the subject and in some embodiments, provide insight as to the rate or pattern of deterioration.

One variable that may be of particular interest is Hb since a decrease in Hb concentration can indicated loss of red blood cells (RBCs) from the circulation and comprised capacity to carry oxygen. RBC loss can in turn be used to assess bleeding (haemorrhage) and haemodynamic optimisation. By knowing the time-dependent change in the subject's Hb profile, (correcting for fluid type and concentration added to the subject's circulation) it is possible for the processing means 601 to calculate the net effect of f blood or other fluid added to the intravascular Hb concentration. In this way, a decrease in measured Hb when compared with the expected Hb count can support a diagnosis consistent with haemorrhage. In one embodiment, processing means 601 comprises part of a closed loop control interface to infusion pumps which controls those pumps to administer therapy for correcting a deficiency in one or more of the monitored variables. This exploits the ability to correlate statistics from a measured variable (Hb) with statistics of therapy (fluid administration. It is to be understood that reference to Hb and fluid administration is only one example of how the principles of the present invention may be applied.

These analyses enable a clinician to detect e.g. if there is bleeding and if so, how rapidly the patient is losing blood from the circulation. This may enable surgeons to determine if there is internal bleeding prior to concluding surgery, saving on emergency re-operation and avoiding potentially life-threatening circumstances. It may also provide the possibility to establish if bleeding is from the venous or arterial circulation and assess if the bleed is settling, stable or worsening. Programing the processing means 601 to estimate the kinetics of volume loss of different fluids from the circulation, and compensating that estimation for the dilution effect of these fluids on the subject's blood enables the system to quantify RBC loss. Of course it is necessary to take account of diuresis and interstitial fluid loss to restore blood volume. If the rate and volume of fluid having a known pharmacodynamic profile is appropriately interfaced to the processing means then it potentially becomes possible to ascertain the extent of RBC loss before concluding surgery. This, combined with other variables monitored according to the invention gives rise to improved outcomes.

In one embodiment, the computer program product includes instructions causing the processing means 601 to interrogate a database in order to identify a course of action targeted at restoring one or more of the haemodynamic variables toward a reference value and more preferably, targeted at restoring normo-volaemic condition in the subject. The database, e.g. database 604, may index haemodynamic variables and a range of therapeutic effectors which influence the behaviour of those haemodynamic variables in the circulation. Once the processing means 601 has identified a course of action it generates a user interface signal configured to cause user interface 603 to present the identified course of action to a user. Typically this involves displaying a message on a display means 606 and/or providing an audible alert or message.

Various devices may be used to measure from the subject the haemodynamic variables required as inputs to the data interface 602. One suitable device is a Vigileo Monitor (Edwards Life Sciences). This monitor provides various indicators of the circulation such as HR, SV, SW and SVR. Thus, data obtained using that device can be supplied directly to data interface 602 for processing by processing means 601.

In one embodiment, when the processing means 601 identifies the subject as having abnormal volaemic condition, and where that condition has deteriorated to an extent that intervention is required, the instructions automatically cause alert driver 605 to alert a clinician, e.g. by providing a message on display means 606 of user interface 603, providing an audible message and/or sending a paging or SMS alert to a hand held mobile device 608. For the scoring regimes indicated in table 1, a combined score exceeding 8 may cause alert driver 605 to display a message and/or audible signal on user interface/display means 603/606. A combined score exceeding e.g. 9 or 10 may cause the alert driver 605 to send a message to an intensivist carrying the device 608.

In one or more embodiments, the device 608 is also capable of displaying haemodynamic mapping images and/or charted haemodynamic data to facilitate inspection by a clinician while away from the subject, as well (or as an alternative to) providing an alert when the subject's data trends outside of safe territory, as ascertained by the processing means by comparison with reference value(s). Similarly, an alert and/or haemodynamic mappings and/or charted data may be presented on one or more remotely located display devices that need not be mobile. This may provide for expert monitoring and diagnosis by a clinician located at one (e.g. city based) location for a patient who is remotely (e.g. country) located.

The following example of a patient experiencing haemorrhagic shock demonstrates the utility of embodiments of the invention in early detection of intravascular volume variation and more specifically, hypovolaemia.

### EXAMPLE A

### Haemorrhagic Shock

73 yr male post Coronary Bypass Grafting.

The patient arrives in ICU at 2000 hrs. The following day at 0230 hrs, HR is 69 bpm; SV is 91 ml; SVR is 1029; iVR is 890 (i.e. SVR.HR/79.9). At 0330 hrs: Hb 67 g/L is detected. The ICU nurse contacts the resident doctor to authorise administration of blood. At that time, HR increases to 75 bpm; SV is 80 ml; SVR has decreases to 850; 1VR is 850.

At 0630 the ICU nurse advises the resident doctor that the patient is unstable; at that time the patient has a HR of 129 bpm; SV of 43 ml; SVR of 909; and iVR of 1581.

Importantly, the SVR variable shows no significant increase (909 compared with 850 three hours earlier) despite the existence of severe hypovolaemia and acidosis. However, a calculated iVR increase to 1581 (a 78% increase since 0230) signifies a compensatory increase in vascular tone occurring as volume is lost from the circulation. Based on this, it can be seen that iVR provides a more accurate measure for the circulatory resistance to flow, which SVR has been previously thought to measure—but in fact does not.

At 0715 hrs, the intensivist is called. At 0735 hrs the intensivist arrives to find: pH of 7.0; BE of −15; HR of 140 bpm, SV of 43; SVR of 690 and iVR of 1209. The patient is immediately transferred to the operating room (at 0758 hrs) for emergency reopen where 3 litres of blood is found in the patient's chest. The patient arrests during chest opening (0818 hrs) which responds to 1 mg boluses of adrenaline. Nine minutes later (0827 hrs), with the chest open, the patient becomes asystolic, is given internal cardiac massage and ventricular pacing wires attached.

The patient is extubated uneventfully 2 days later and is grossly intact neurologically.

When haemorrhage is occurring (resulting in loss of blood from the circulation), HR rises as SV falls, but CO is maintained. As SV falls, the circulation ‘tenses up’ and iVR rises. Since SVR does not compensate for HR, as the HR rises, the SVR appears to fall, so SVR is not useful as a guide to hypovolaemia.

Cardiac Output (CO) and Central Venus Pressure (CVP) may used as a guide to identifying the existence of hypovolaemia. However, because the body compensates for falling SV by increasing HR, and it compensates for falling SV by increasing iVR, the detection of hypovolaemia using CO and pressure is late and unreliable. In contrast, if the variables HR, SV and iVR are used, and in cases where the patient is mechanically ventilated SW is also used, it becomes possible to identify the existence of abnormal volaemic condition in the subject earlier and with greater reliability. If each of these variables is allocated a score of e.g. 0, 1, 2, 3 depending on the degree of deviation for each variable from a reference value, then it becomes possible to identify the existence of e.g. hypovolaemia on the basis of the change in these variables and more specifically, their deviation from the reference value. This is achievable even though there is no blood coming from the operative drains and other clinical signs of hypovolaemia are not yet present because the condition is not sufficiently advanced.

### EXAMPLE B

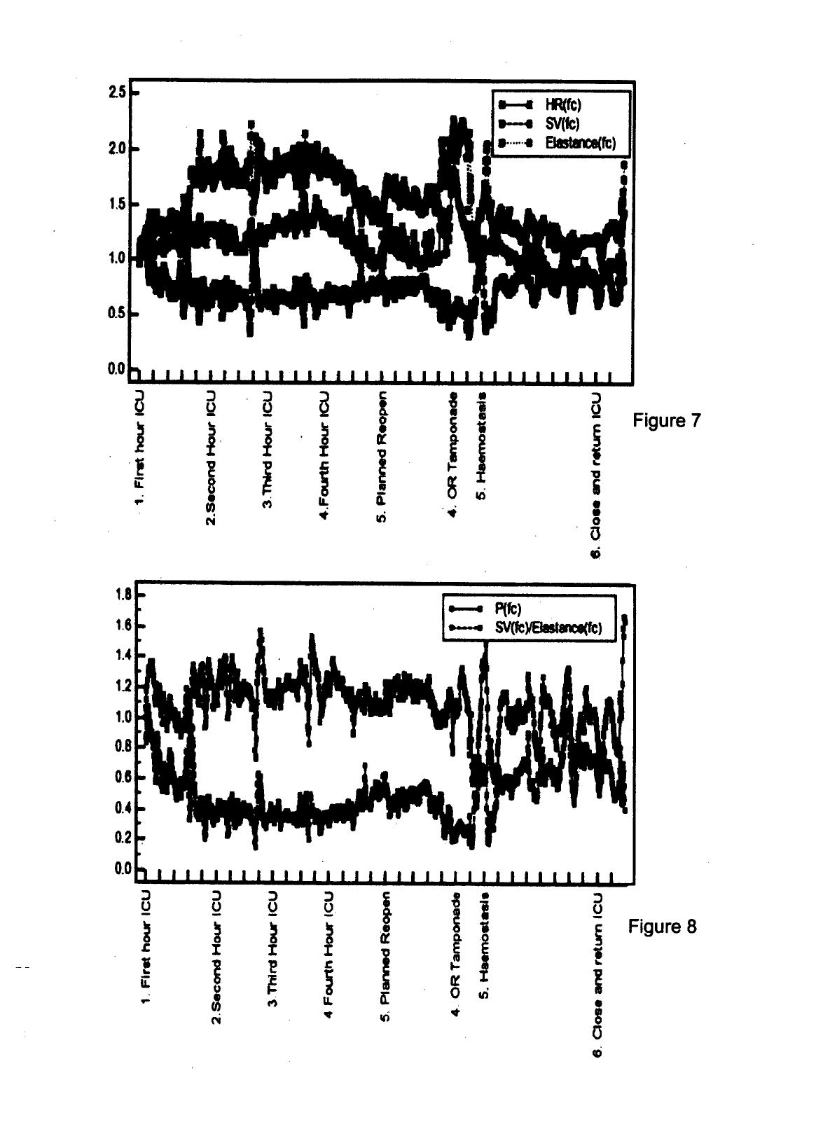

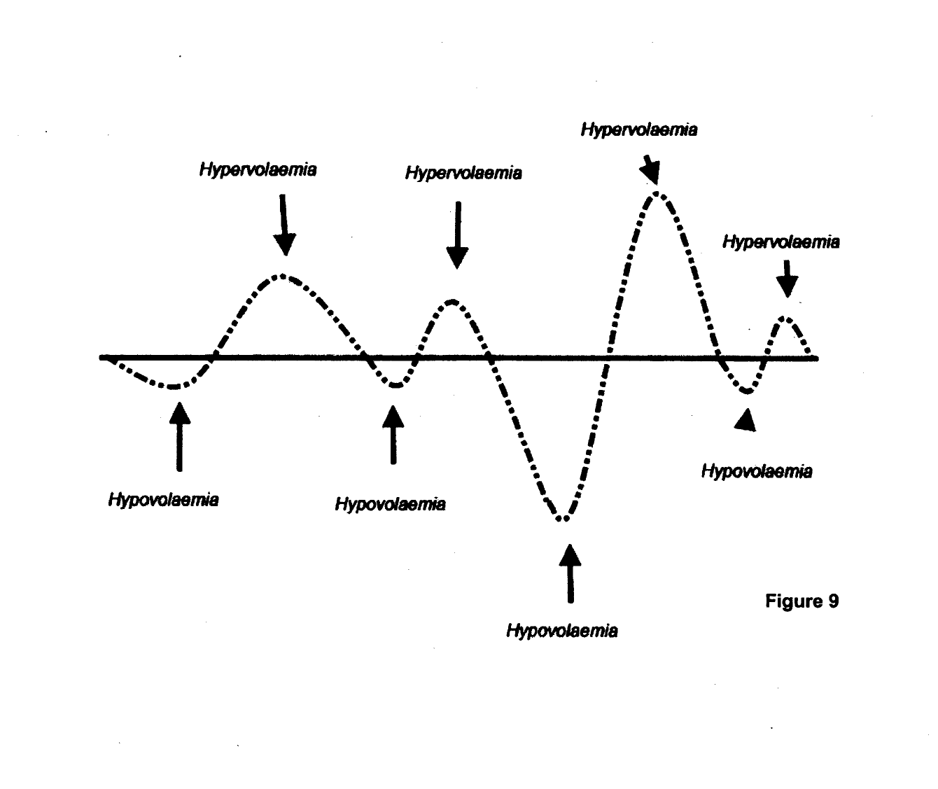

Patient receives surgery for esophagectomy and is “oozy” when closing chest. He is returned to the operating theatre 5 hours after surgery with a suspected haemorrhage, but overly tamponading from concealed haemorrhage within the chest. FIG. 7 illustrates data charting HR_{fc}, SV_{fc} and E_{fc} over time. FIG. 8 illustrates data charting fractional changes in Pressure (P_{fc}) and a ratio of SV_{fc}/E_{fc}. If only the fractional change in pressure is taken into account, there is no indication of the haemorrhage occurring. However, if the ratio SV_{fc}/E_{fc} is considered, it becomes apparent that the subject was experiencing a life-threatening haemorrhage within one hour of surgery, although bleeding was not suspected by clinical staff until 2 to 4 hours after surgery.

The preceding examples highlight advantages of the present invention. Using a “pool” of variables which are known to change with decreased filling of the heart to identify the existence and preferably, extent, of variations in intravascular volume provides clinicians with the ability to identify abnormal volaemic conditions earlier, and to administer therapies sooner than would otherwise be the case. This can lead to improved clinical outcomes. If enough data is collected and analysed, a robust scoring system, based on the pool of variables, can indicate the extent of volaemic abnormality in a reliable and quantitative manner and may be used to trigger clinical intervention. Intervention driven by a score based on actual patient data rather than subjective observations has obvious advantage over the current approaches to identifying the existence of hypovolaemia. In addition, increasing the number of variables in the pool increases the sensitivity of the score.

It is hypothesised that improvements in clinical outcomes will not only arise by reducing the likelihood of hypovolaemic shock, but also by avoiding possible vessel damage caused by cyclical variation in intravascular volume. FIG. 7 is a schematic illustration of cyclical volume changes created when clinicians treat abnormal volaemic states based on traditional indicators of hypo- and hyper-volaemic. The inventor hypothesises that this cyclical variation leads to overdistension of the circulation because clinicians have no way of detecting or measuring under-filling (or adequate filling) of the circulation. Thus, when correcting a state of hypovolaemia, clinicians can inadvertently overfill, over compensating for the estimated pre-load deficiency. Overdistension of the circulation can cause fluid to leak out of the circulation causing interstitial oedema and/or fluid overload.

The methods, systems and computer program product of the present invention provide an alternative approach to monitoring volume changes in the circulation which may obviate or at least ameliorate problems associated with overfilling and overdistension of the circulation.

The present application relates to visual mapping techniques such as those described in International patent application PCT/AU2010/000748, the entire contents of which are hereby incorporated herein by reference.

By combining the novel and inventive approaches to monitoring and representing visually, the state of the circulation, clinical outcomes can be improved markedly. Ideally, a visual display which shows, in real time, haemodynamic variables measured from a subject and also shows a reference value or range of values that is “normal” for a particular variable enables calculation (automatically by a computing device) or estimation (by inspection of the visual mapping) of a deviation from normal. Reference values may be subject specific or may be obtained from a population of individuals and can be used to identify and quantify the level of abnormality in the subject's condition. This gives rise to improved therapies that can be administered in response to a deviation from normal/reference values. Moreover, the effect of therapies can be monitored in real time and this can be used to individualise treatment, as well as to recognise when a pathological state is present and/or assess the efficacy of various therapies.

It is to be understood that various modifications, additions and/or alterations may be made to the parts previously described without departing from the ambit of the present invention as defined in the claims appended hereto.

1. A method for processing data to monitor volaemic condition in a human or animal subject, including the steps of:
(a) identifying one or more reference values for each of one or more haemodynamic variables;
(b) receiving haemodynamic data representing the one or more haemodynamic variables measured from the subject over time;
(c) for at least one of the one or more haemodynamic variables, comparing the haemodynamic data with the respective one or more reference values; and
(d) identifying the existence of abnormal volaemic condition in the subject when the comparison indicates a deviation from the one or more reference values for at least one of the haemodynamic variables.

2. A method according to claim 1 wherein the haemodynamic variables include one or more of Heart Rate (HR), Vascular Resistance, Elastance (E) and Stroke Volume (SV).

3. A method according to claim 2 wherein Vascular Resistance is adjusted by a multiplier to correct for heart rate effects, giving rise to instantaneous vascular resistance (iVR) as a haemodynamic variable.

4. A method according to claim 1 wherein the haemodynamic variables include one or more of Central Venous Oxygen Saturation (ScvO2), Haemoglobin (Hb), Systolic Blood Pressure (SBP), Diastolic Blood Pressure (DBP), Pulse Pressure (PP) and Mean Arterial Pressure (MAP).

5. A method according to claim 1 wherein the one or more reference values for at least one of the one or more haemodynamic variables is derived from actual haemodynamic data obtained from the subject.

6. A method according to claim 1 wherein the one or more reference values is determined from data obtained from a population of individuals.

7. A method according to claim 1, including the step of displaying on a display means:
a time-based chart representing received data values for at least one of the one or more haemodynamic variables; and
optionally, an indicator for the respective one or more reference values.

8. A method according to claim 1, including the step of:
calculating a ratio of received data value to reference value for at least one haemodynamic variable; and
displaying the calculated ratio on a display means.

9. A method according to claim 1, including one or more of:
(a) allocating a score for at least one of the haemodynamic variables in the data, the allocated score representing a degree of deviation from one or more reference values for that haemodynamic variable;
(b) determining an extent of abnormal volaemic condition according to one or more allocated scores; and
(c) determining an extent of abnormal volaemic condition according to a combined score from the allocated scores.

10. (canceled)

11. A method according to claim 8 including the step of determining a volaemic index by:
(i) calculating a sum total of:
(a) each ratio value calculated for each haemodynamic variable known to decrease with volume loss; plus
(b) the inverse of each ratio value calculated for each haemodynamic variable known to increase with volume loss; and
(ii) dividing the calculated sum total by the number of haemodynamic variables considered in step (i).

12. A method according to claim 9 including one or more of:
(a) determining a reliability indicator for indicating the reliability of an allocated score, wherein an allocated score calculated using more variables has a higher reliability than a score calculated using fewer variables;
(b) adjusting an allocated score to have greater or lesser influence on determining the extent of abnormal volaemic condition, wherein the adjustment is based on one or more of:
(i) sensitivity of a haemodynamic variable to volume changes;
(ii) age, fitness, gender, weight or condition of the subject; and
(iii) therapies being administered to the subject;
(c) adjusting an allocated score to have greater or lesser influence on determining the extent of abnormal volaemic condition, wherein the adjustment is a weighting coefficient which is determined automatically by a processing device using reference data from a population of individuals, and subject-specific data provided to the processing device;
(d) adjusting an allocated score to have greater or lesser influence on determining the extent of abnormal volaemic condition, wherein the adjustment is a weighting coefficient which is determined on a case by case basis by a clinician; and
(e) adjusting an allocated score to have greater or lesser influence on determining the extent of abnormal volaemic condition, wherein the adjustment uses a hybrid of (c) and (d).

13.-16. (canceled)

17. A method according to claim 1 including the step of selecting automatically a course of action targeted at restoring one or more of the haemodynamic variables toward the one or more reference values by interrogating a collection of pooled data indexing haemodynamic variables and therapeutic effectors obtained from a population of individuals.

18. A non-transitory computer readable medium storing a computer program comprising instructions for controlling a processing means to execute a process for monitoring an abnormal volaemic condition in a human or animal subject, wherein the instructions cause the processing means to:
(a) receive one or more reference values for each of one or more haemodynamic variables;
(b) receive haemodynamic data representing the one or more haemodynamic variables measured from the subject over time;
(c) for at least one of the one or more haemodynamic variables, compare the haemodynamic data with the respective one or more reference values; and
(d) identify the existence of abnormal volaemic condition when the comparison indicates a deviation from a reference value for at least one of the haemodynamic variables.

19. A non-transitory computer readable medium according to claim 18 storing a computer program including instructions causing a processing means to calculate one or more reference values for at least one of the one or more haemodynamic variables using actual haemodynamic data previously obtained from the subject.

20. A non-transitory computer readable medium according to claim 18 storing a computer program wherein the instructions cause the processing means to receive one or more reference values from memory means in communication with the processing device, wherein the one or more reference values has been determined using data obtained from a population of normo-volaemic individuals.

21. A non-transitory computer readable medium according to claim 18 storing a computer program, wherein the instructions cause the processing means to generate a display signal for causing a display means to present:
a time-based chart of data values representing at least one of the one or more haemodynamic variables; and
optionally, an indicator for the respective one or more reference values.

22. A non-transitory computer readable medium according to claim 18 storing a computer program, wherein the instructions cause the processing means to perform steps including one or more of:
(a) calculating, for at least one haemodynamic variable, a ratio of actual data value to one or more reference values displaying the calculated ratio on a display means;.
(b) calculating a score for at least one of the haemodynamic variables in the data, the calculated score representing a deviation of a data value from a reference value and estimating an extent of abnormal volaemic condition according to one or more calculated scores; and
(c) calculating a score for a plurality of haemodynamic variables and estimating an extent of abnormal volaemic condition by combining the calculated scores.

23.-24. (canceled)

25. A non-transitory computer readable medium according to claim 22, storing a computer program wherein the instructions cause the processing means to apply a weighting coefficient to one or more of the calculated scores, wherein the weighting coefficient is selected for a particular haemodynamic variable based on one or more of:
sensitivity of a haemodynamic variable to volume changes;
age, fitness, gender, body mass or physiological condition of the subject; and
a therapy being administered to the subject.

26. A non-transitory computer readable medium according to claim 25 storing a computer program, wherein instructions cause the processing means to select automatically, the weighting coefficient applicable to a haemodynamic variable.

27. A non-transitory computer readable medium according to claim 25 storing a computer program, wherein the instructions cause the processing means to receive an input from a clinician which indicates the weighting coefficient applied to a score calculated for a haemodynamic variable.

28. A non-transitory computer readable medium according to claim 18 storing a computer program, wherein the instructions cause the processing means to interrogate a database indexing haemodynamic variables and therapeutic effectors and identify a course of action targeted at restoring one or more of the haemodynamic variables toward a reference value, and generating a user interface signal configured to cause a user interface to present the identified course of action to a user.

29. A method of representing visually a state of the circulation of a human or animal subject, including the steps of:
(a) charting on a display means in substantially real time, data obtained from the subject representing one or more haemodynamic variables of the subject; and
(b) charting on the display means a reference value indicative of substantially normal values for one or more of the charted haemodynamic variables.

30. A method according to claim 29 wherein charting the data obtained from the subject includes presenting time-elapsed changes in the data values.

31. A method according to claim 29 including the step of calculating deviation of the data obtained from the subject from a relevant reference value and optionally, charting the calculated deviation on the display means.

32. (canceled)

33. A method according to claim 31 wherein the deviation is calculated as an average or a rolling average of values occurring in a finite time period.

34. A system for monitoring an abnormal volaemic condition in a human or animal subject, the system including:
(a) at least one processing means for executing steps according to the method of claim 1;
(b) a user interface including display means for displaying haemodynamic data and/or a ratio of actual to reference values for one or more haemodynamic variables; and
(c) a data interface for receiving data representing the one or more haemodynamic variables measured from the subject.

35. A system according to claim 34 including an alert driver for generating an alarm or communicating an alert when a score exceeds a threshold value.

36.-38. (canceled)

39. A system for monitoring an abnormal volaemic condition in a human or animal subject, the system including:
(a) at least one processing means for executing the computer program stored by the non-transitory computer readable medium of claim 18;
(b) a user interface including display means for displaying haemodynamic data and/or a ratio of actual to reference values for one or more haemodynamic variables; and
(c) a data interface for receiving data representing the one or more haemodynamic variables measured from the subject.

40. A system according to claim 39 including an alert driver for generating an alarm or communicating an alert when a score exceeds a threshold value.
